(19) United States
(12) Patent Application Publication
(10) Pub. No.: US 2006/0210482 A1
(43) Pub. Date: Sep. 21, 2006

(54) Chemical composition and method for cold and sinus relief

(76) Inventor: John Cassara, Sierra Madre, CA (US)

Correspondence Address: TROJAN LAW OFFICES, 9250 WILSHIRE BLVD, SUITE 325, BEVERLY HILLS, CA, 90212, US

(21) Appl. No.: 11/312,871

(22) Filed: Dec. 20, 2005

Related U.S. Application Data
(63) Continuation-in-part of application No. 11/018,681, filed on Mar. 18, 2005, now abandoned.

(51) Int. Cl. A61L 9/04; A61K 31/60; A61K 31/045; A61K 31/05

(52) U.S. Cl. 424/45; 514/159; 514/729; 514/724; 514/731

(57) ABSTRACT

A chemical composition and method for treatment of the common cold, sinusitis and other conditions associated with nasal congestion is provided. The chemical composition is made of predetermined concentrations of thymol, alcohol, eucalyptol, menthol, methyl salicylate, a preservative, saline solution and a buffer. The method includes the step of administering the chemical composition of the invention via either intranasal instillation, oral inhalation or intermittent positive pressure breathing. Administration of the chemical composition via the method of the invention clears the nasal passages and alleviates the symptoms of the common cold, sinusitis, and related conditions.

The present application is a continuation-in-part of U.S. patent application Ser. No. 11/018,681, filed Mar. 18, 2005, the entire contents of which are hereby incorporated by reference and relied upon.

### FIELD OF THE INVENTION

This invention relates generally to a chemical composition and a method, and more particularly, to a chemical composition and method for relieving the symptoms of the common cold, sinusitis and other related conditions associated with nasal congestion.

### BACKGROUND OF THE INVENTION

The common cold (also known as “Nasopharyngitis”) is a mild viral infectious disease of the nose and throat. Some of the typical symptoms include sneezing, sniffling, running/blocked nose (often these occur simultaneously, or one in each nostril). Additional symptoms may include scratchy, sore, or phlegmy throat, coughing, headache and tiredness. Colds typically last three to five days, with residual coughing lasting up to three weeks. As its name suggests, the cold is the most common of all human diseases, infecting subjects at an average rate of slightly over one infection per year per person. Infection rates greater than three infections per year per person are not uncommon in some populations.

Sinusitis is the inflammation (bacterial, viral, allergic or autoimmune) of the paranasal sinuses. Chronic sinusitis is one of the most typical complications of the common cold. Symptoms of sinusitis include nasal congestion, facial pain, headache, fever, and general malaise.

There is no known cure for the common cold, i.e., there is no treatment that directly fights the virus. Available treatments therefore focus on relieving the symptoms. A variety of nasal sprays are currently available as medicine for the common cold. These sprays include nasal decongestants, steroids, and seawater.

Therapeutic measures for sinusitis include inhaling steam, nasal irrigation using a warm saline solution and over-the-counter decongestants. If sinusitis doesn't improve within 48 hours, or is causing significant pain, doctors typically prescribe antibiotics or nasal steroids.

Decongestants reduce swelling of the mucous membrane in the nose and sinuses associated with sinusitis by constricting blood vessels and reducing the blood supply to nasal mucous membranes. This reduces nasal congestion, stuffiness, and runny noses. Typically used nasal decongestants include ephedrine, oxymetazoline, phenylephrine, pseudoephedrine, xylometazoline, tetrahydrozoline or neosynephrine. A major disadvantage of decongestants is that most of them are associated with serious side effects. Such side effects may include: increased blood pressure, dizziness, nervousness or irritability, insomnia, and ciliostasis.

In addition to decongestants, there is a wide variety of commercially available nasal steroids. Some nasal steroids available in spray form are: Vancenase and Beconase (beclomethasone), Flonase (fluticasone), Rhinocort (budesonide), Nasonex (mometasone) and Nasacort (triamcinolone). Similarly to the nasal decongestants, these steroid sprays have serious side effects. Such side effects may include: allergic reactions, nosebleeds, septum perforation, mucosal damage and loss of smell.

Further, seawater or saline sprays are used to help relieve nasal congestion. Saline nasal sprays are typically used to loosen mucus in the nose and help to wash it out. They are generally considered safe no matter how long they are used or how often. Additionally, there are known saline solutions containing iodine used to treat nasal congestion, as described in U.S. Pat. No. 5,897,872 (Picciano). However, saline nasal sprays are not very effective in relieving nasal congestion due to the common cold and salt water frequently causes discomfort to the user. Additionally, iodine in liquid and vapor form is generally known to be quite irritating to human eyes and mucous membranes.

Accordingly, there is a need for a chemical composition and a method for effectively treating symptoms of the above-referenced conditions, while overcoming the disadvantages of the prior art.

### SUMMARY OF THE INVENTION

The present invention discloses an iodine-free chemical composition and a method for the treatment of the common cold, sinusitis and related conditions associated with nasal congestion. The chemical composition comprises thymol (0.01-0.1% by weight), alcohol (1-90% by weight), eucalyptol (0.01-0.1% by weight), methyl salicylate (0.01-0.1% by weight), menthol (0.01-0.1% by weight), a preservative, saline solution and a buffer.

In the preferred embodiment of the invention, the alcohol is ethyl alcohol. In another embodiment of the invention, the chemical composition additionally comprises phenol (0.01-2% by weight).

According to the method of the present invention, the composition of the present invention can be administered to a user via various routes. In the preferred embodiment, the chemical composition is administered via intranasal instillation. In additional embodiments, the chemical composition may be administered via oral inhalation or intermittent positive pressure breathing. Administration of the chemical composition of the invention provides safe and effective relief for nasal congestion associated with common cold, sinusitis and the like conditions.

### DETAILED DESCRIPTION OF THE INVENTION

The invention is a chemical composition and method for alleviating the symptoms of the common cold, sinusitis and various other conditions associated with nasal congestion.

The chemical composition of the invention comprises thymol (0.01-0.1% by weight), alcohol (1-90% by weight), eucalyptol (0.01-0.1% by weight), methyl salicylate (0.01-0.1% by weight), menthol (0.01-0.1% by weight), a preservative, saline solution and a buffer. In accordance with the principles of the present invention, the chemical composition is iodine-free and thereby avoids the above-described disadvantages of the prior art.

The preferred chemical composition contains thymol in the amount of 0.01-0.1% by weight. Thymol is a well-known essential oil which is commonly utilized for its antimicrobial activity in a variety of preparations, including those in oral hygiene.

The preferred chemical composition also contains alcohol in the amount of 1-90% by weight. Generally speaking, the term alcohol almost always refers to ethanol and often to any beverage that contains ethanol. Other forms of alcohol are usually described with a clarifying adjective, as in isopropyl alcohol or by the suffix -ol, as in isopropanol. In chemistry, alcohol is a more general term, applied to any organic compound in which a hydroxyl group (—OH) is bound to a carbon atom, which in turn is bound to other hydrogen and/or carbon atoms. In the preferred embodiment of the present invention, ethyl alcohol is used. However, one of ordinary skill in the art will appreciate that other suitable alcohols may be used in alternative embodiments of the invention.

The preferred embodiment of the invention also contains eucalyptol in the amount of 0.01-0.1% by weight. Eucalyptol is a natural organic compound which is a colorless liquid. Eucalyptol comprises about 85 percent of the essential oil of eucalyptus, hence the common name of the compound. Because of its pleasant spicy aroma and taste, eucalyptol is often used in flavorings, fragrances, and cosmetics. Eucalyptol is also an ingredient in many commercially available brands of mouthwash and cough suppressants. Additionally, eucalyptol is commonly known to be capable of reducing inflammation and pain.

In the preferred embodiment of the invention, the chemical composition also contains Methyl Salicylate, 0.01-0.1% by weight. Methyl Salicylate (also known as salicylic acid methyl ester, oil of wintergreen, betula oil, methyl ester) is a natural product of many species of plants. Methyl salicylate is routinely used as an ingredient in deep heating rubs, and in small amounts as a flavoring agent. Additionally, Methyl Salicylate is a well known antiseptic.

In the preferred embodiment of the invention, the chemical composition also contains Menthol, 0.01-0.1% by weight. Menthol is a covalent organic compound made synthetically or obtained from peppermint or other mint oils. It is a waxy, crystalline substance, clear or white in color, which is solid at room temperature and melts slightly above. Menthol is widely recognized as having local anesthetic and counterirritant qualities, and it is widely used to relieve minor throat irritation.

The preferred embodiment of the invention also contains a preservative. A preservative is a natural or synthetic chemical that is added to products such as foods, pharmaceuticals, paints, biological samples, etc., to retard spoilage, whether from microbial growth, or undesirable chemical changes. A distinction is sometimes made between anti-microbial preservatives which function by inhibiting the growth of insects, bacteria and fungi, and antioxidants, which inhibit the oxidation of food constituents. Common anti-microbial preservatives include sodium nitrate, sodium nitrite, sulfites, (sulfur dioxide, sodium bisulfate, potassium bisulfate, etc.) and disodium EDTA. One preferred embodiment of the invention contains phenylcarbinol (benyl alcohol) and benzalkonium chloride as preservatives. However, it will be appreciated by one of ordinary skill in the art that a variety of commonly used preservatives, including bisulfates, can be used in the chemical composition of the invention.

The preferred embodiment of the present invention also contains a buffered saline solution. Buffer solutions are solutions which resist change in pH upon addition of small amounts of acid or base. Buffer solutions usually consist of either a weak acid and its salt or a weak base. The present invention preferably contains phosphate buffered saline solution.

Phosphate buffered saline is a commonly used buffer solution. It is a salty solution containing sodium chloride, sodium phosphate and potassium phosphate. The buffer helps to maintain a constant pH. The concentration of phosphate buffered saline is typically chosen such that the saline is isotonic and non-toxic to human cells. Although phosphate buffered saline is used in the preferred embodiment of the present invention, one of ordinary skill in the art will appreciate that any other buffered saline solution may be used. Alternatively, a plain saline solution may be used and a buffer may be separately added.

In another embodiment, the chemical composition additionally contains phenol, 0.1-2.0% by weight. Phenol, also known under the old name carbolic acid, is a colorless crystalline solid with a typical sweet tarry odor. Its structure is that of a hydroxyl group (—OH) bonded to a phenyl ring. Thus, phenol is an aromatic compound.

The following specific Examples are used to illustrate the preferred embodiments of chemical composition of the present invention.

### EXAMPLE 1

Formulation for Intranasal Instillation:

Solution 1-15 ml of Moisturizing Saline Nasal Spray, containing Sodium Chloride (0.65% by weight) buffered and made isotonic with Sodium Bicarbonate, also containing phenylcarbinol (benyl alcohol) and benzalkonium chloride as preservatives.

Solution 2-45 ml of solution containing: thymol 0.064% by weight, eucalyptol 0.092% by weight, methyl salicylate 0.060% by weight, menthol 0.042% by weight, alcohol 21.6% by volume, water, sorbitol solution, flavoring, poloxamer 407, benzoic acid, sodium benzonate, sodium saccharin.

Solutions 1 and 2 are mixed together to make the chemical composition of the invention acceptable for administration via intranasal instillation. Once the two solutions are mixed, the final concentrations are: Thymol 0.048%, Eucalyptol 0.069%, methyl salicylate 0.045%, menthol 0.0315%, alcohol 16.2%.

### EXAMPLE 2

Formulation for oral inhalation: thymol 0.064% by weight, eucalyptol 0.092% by weight, methyl salicylate 0.060% by weight, menthol 0.042% by weight, alcohol 21.6% by volume, water, sodium saccharin, sodium benzonate, sorbitol solution, flavoring, polyxamer 407. This solution forms the chemical composition of the invention acceptable for administration via oral inhalation.

### EXAMPLE 3

Formulation for Intermittent Positive Pressure Breathing:

Solution 1: 1 ml of absolute 100% ethyl alcohol;

Solution 2: 7 ml of oral inhalation solution identified in Example 2 (thymol 0.064% by weight, eucalyptol 0.092% by weight, methyl salicylate 0.060% by weight, menthol 0.042% by weight, alcohol 21.6% by volume, water, sodium saccharin, sodium benzonate, sorbitol solution, flavoring, polyxamer 407).

Solutions 1 and 2 are mixed together to form the chemical composition of the invention acceptable for administration via IPPB.

The present invention further provides a method of treating nasal congestion caused by common cold, sinusitis or the like conditions. The method includes the step of administering the above-described chemical composition to a user in need. The chemical composition can be administered via several different routes. The concentration of the constituents in the chemical composition of the invention is varied for each method of administration.

In the preferred embodiment, the method of administration is intranasal instillation. Intranasal instillation is well known in the art and represents applying a liquid form of chemical composition directly into the nostrils.

In accordance with this method of the invention, the chemical composition of the invention is produced in its liquid form. The chemical composition is then packaged in a commonly known applicator bottle. Such a bottle typically includes a nozzle, an opening or the like construction, allowing a user to apply the chemical composition from the bottle to a nostril either by spraying a liquid mist or drop by drop.

Preferably, a spray bottle is used. Once a patient sprays the chemical composition of the present invention into each nostril, the chemical composition is absorbed by the mucosal membranes in the nostrils. A preferred chemical composition made for use with this method of the invention corresponds to the composition described in EXAMPLE 1 above.

In another embodiment, the method of administration of the chemical composition of the invention is oral inhalation. In accordance with this method, the patient breathes in the chemical composition in vaporized form. Preferably, a hand-held nebulizer is used. A nebulizer is a device used to administer medication to people in the form of a liquid mist to the airways. Typically, a nebulizer pumps air or oxygen through a liquid medicine to turn it into a vapor, which is then inhaled by the patient.

Alternatively, the composition of the invention may be breathed in as a vapor from a boiling solution. In this embodiment, the chemical composition is made in its liquid form and boiled, releasing vapor. It will be appreciated by one of ordinary skill in the art that the chemical composition of the invention may also be made in powder form and mixed with water, which would then be boiled, releasing the medicinal vapor.

Once the chemical composition of the invention in vapor form is inhaled by a patient, it is absorbed by the patient's lungs. A preferred chemical composition made for use with this method of the invention corresponds to the composition described in EXAMPLE 2 above.

In another embodiment, the method of administration of the chemical composition of the invention is intermittent positive pressure breathing (also known as IPPB). IPPB is a technique used to provide short-term or intermittent mechanical ventilation for the purpose of augmenting lung expansion, delivering aerosol medication, or assisting ventilation. IPPB is not a therapy of first choice for aerosol delivery or lung expansion in spontaneously breathing patients when other less expensive and less invasive therapies can reliably meet clinical objectives. Typically, IPPB includes volume-, pressure-, time-limited, or flow-cycled ventilation. Generally, IPPB may be applied to intubated as well as non-intubated patients. IPPB is typically administered through a mouthpiece or by mask and is administered by a licensed respiratory therapist.

In accordance with this method, the patient breathes in the chemical composition of the invention in vapor form through the mouthpiece or mask of the IPPB machine. Typically, the IPPB machine helps the patient take a deep breath by pushing air in when the patient breathes. Just like a nebulizer, the IPPB machine can use oxygen instead of air. Once the vapor is inhaled, the chemical composition of the present invention is absorbed through the patient's lungs. A preferred chemical composition made for use with this method of the invention corresponds to the composition described in EXAMPLE 3 above.

The following specific Examples are used to illustrate the operation and efficacy of the method of the present invention in treating nasal congestion associated with the common cold, sinusitis, and the like disorders.

### EXAMPLE 4

Twenty volunteers suffering from symptoms including nose congestion, runny nose, sore throat, cough, sneezing, sinus congestion, headache, etc., were treated with the chemical composition of the present invention using the nasal instillation method. Specifically, the individuals administered the chemical composition of the present invention to themselves using a spray bottle.

Some volunteers sprayed once into each nostril and some individuals sprayed twice into each nostril. Some individuals sprayed the chemical composition of the invention only one time, and some individuals sprayed 2-3 times daily.

Each of the twenty volunteers reported that the nasal congestion was relieved after just one application of the chemical composition of the invention. Some volunteers reported that their sore throat was relieved. Some volunteers reported that their congested nasal passages completely opened up. Some volunteers reported that their runny noses completely stopped running. Additionally, some volunteers reported that their symptoms were alleviated or completely gone within 1 hour of the administration of the chemical composition.

It was also found by the inventor that after the onset of the cold, administration of the chemical composition of the invention via only intranasal instillation sufficiently works to completely relieve a patient from symptoms. However, subsequent to 48 hours from the onset of the cold, the chemical composition of the invention is more effective in alleviating the symptoms if administered via both the intranasal instillation and the oral inhalation method. Additionally, it was found by the inventor that the chemical composition of the invention effectively and safely treats nasal congestion when administered via intranasal instillation between one and four times a day, as needed to relieve symptoms.

### EXAMPLE 5

Three volunteers suffering from bronchitis and pulmonary congestion were administered the chemical composition of the present invention through the Intermittent Positive Pressure Breathing method, using the solution as described in Example 3 above. During the IPPB, 5 ml of solution from Example 3 was being mixed with oxygen at 5 liters/minute and the individuals were administered the chemical composition of the present invention in a single treatment lasting approximately five minutes.

After treatment, all three volunteers experienced significantly improved breathing with less wheezing. Additionally, the three volunteers reported an expectorant effect (discharge or expulsion of tracheal or bronchial mucus through respiratory tract) for approximately 20 minutes after the treatment. The effect from the administration of the chemical composition lasted approximately 4-6 hours before congestion returned in volunteers to a lesser degree than before. None of the volunteers reported any side effects.

While the invention has been described in connection with what is presently considered to be the most practical and preferred embodiments, it is to be understood that the invention is not limited to the disclosed embodiments, but on the contrary is intended to cover various modifications and equivalent arrangements included within the spirit and scope of the appended claims.

1. A chemical composition for treatment of nasal congestion comprising:
thymol, 0.01-0.1% by weight;
alcohol, 1-90% by volume;
eucalyptol, 0.01-0.1% by weight;
methyl salicylate, 0.01-0.1% by weight;
menthol, 0.01-0.1% by weight;
a preservative;
a saline solution; and
a buffer,
wherein said chemical composition is essentially iodine-free.

2. The chemical composition of claim 1, further comprising phenol, 0.1-2% by weight.

3. The chemical composition of claim 1, wherein said alcohol is ethyl alcohol.

4. The chemical composition of claim 1, wherein said saline solution is phosphate buffered saline.

5. A method of treatment of symptoms of medical conditions associated with nasal congestion, comprising the step of administering a chemical composition to a user, said composition comprising:
thymol, 0.01-0.1% by weight;
alcohol, 1-90% by volume;
eucalyptol, 0.01-0.1% by weight;
methyl salicylate, 0.01-0.1% by weight;
menthol, 0.01-0.1% by weight;
a preservative;
a saline solution; and
a buffer.

6. The method of claim 5, wherein said composition further comprises phenol, 0.1-2% by weight.

7. The method of claim 5, wherein said composition is administered via intranasal instillation.

8. The method of claim 7, wherein a spray bottle is used to administer said composition to the user's nostril.

9. The method of claim 5, wherein said composition is administered via oral inhalation.

10. The method of claim 9, wherein a hand held nebulizer is used to administer said composition.

11. The method of claim 5, wherein said composition is administered via intermittent positive pressure breathing.
